(19) United States
(12) Patent Application Publication
(10) Pub. No.: US 2024/0390133 A1
(43) Pub. Date: Nov. 28, 2024

(54) ELECTROSPUN TISSUE REPAIR SCAFFOLDS WITH SUTURE MANAGEMENT

(71) Applicants: NFS IP Holdings, LLC, Dublin, OH (US); Atreon Orthopedics LLC, Columbus, OH (US)

(72) Inventors: Jed K. JOHNSON, London, OH (US); Ronald Lloyd BRACKEN, Monroe, GA (US); Jason CHAKROFF, Columbus, OH (US); Joseph HUDDLE, Columbus, OH (US); Carly SCHWARTZ, Columbus, OH (US); Kelly HENDRIX, Scottsdale, AZ (US); Steven OGG, Lancaster, OH (US); Eric NORDSTROM, Manalapan, NJ (US)

(73) Assignees: NFS IP Holdings, LLC, Dublin, OH (US); Atreon Orthopedics LLC, Columbus, OH (US)

(21) Appl. No.: 18/673,735

(22) Filed: May 24, 2024

Related U.S. Application Data
(60) Provisional application No. 63/468,919, filed on May 25, 2023.

(51) Int. Cl. A61F 2/08; A61L 27/26

(52) U.S. Cl. CPC A61F 2/08; A61L 27/26; A61F 2220/005; A61F 2220/0075; A61F 2240/001; A61F 2250/0067; A61L 2430/10

(57) ABSTRACT

An electrospun onlay for rotator cuff repairs and related procedures. The onlay includes an aperture and a conduit extending towards the aperture, wherein a longitudinal axis of the conduit is aligned with the aperture. The aperture and the conduit are configured to receive one or more surgical sutures therethrough. The scaffold is electrospun from one or more biocompatible polymers. In use, the onlay serves as a tissue growth scaffold to facilitate tendon-bone reattachment.

### CROSS-REFERENCE TO RELATED APPLICATIONS

The present application claims priority to U.S. Provisional Patent Application No. 63/468,919, titled ELECTROSPUN TISSUE REPAIR SCAFFOLDS WITH SUTURE MANAGEMENT, filed May 25, 2023, which is hereby incorporated by reference herein in its entirety.

### BACKGROUND

When soft tissue (e.g., a tendon or ligament) tears or ruptures and pulls away from the bone to which it is attached, surgery is necessary. In a typical reconstruction surgery, such as a rotator cuff or Achilles tendon repair, a suture and suture anchor are typically used to secure the soft tissue back to the bone. Once it is “reattached” to the bone, the soft tissue and bone are expected to heal to reform a strong bone-soft tissue interface. Frequently, however, such healing is suboptimal or is at risk for re-rupture during the healing period. Therefore, a need exists to decrease the healing time and improve the quality of the repair of the soft tissue to the bone to more closely approximate the strength and quality seen at an uninjured bone-soft tissue interface.

Scaffolds may be utilized to improve tissue healing in rotator cuff tear repair procedures; however, the scaffolds must be in tight contact with the bone and soft tissue in order to properly promote healing. In practice, conventional tissue repair scaffolds used in rotator cuff repair procedures may not stay in intimate contact with the bone and/or soft tissue due to the intraoperative challenges with suturing the scaffolds in place. Approximately 450,000 rotator cuff repairs are performed each year, which is an increase of 14% in the last decade. Therefore, the number of individuals that could benefit from improved healing from such procedures is steadily increasing. Accordingly, scaffolds having improved designs that allow them to be better secured to the patient's anatomy would be highly beneficial in the technical field and would benefit a large number of individuals.

### SUMMARY

The present disclosure is directed to electrospun devices for improving healing at interfaces between bone and soft tissue. In one particular embodiment, the present disclosure is directed to a device for assisting surgeons in intraoperatively managing and organizing surgical sutures in combination with biocompatible scaffolds.

In one embodiment, the present disclosure is directed to a graft comprising an aperture and a conduit extending towards the aperture. A longitudinal axis of the conduit is aligned with the aperture. The aperture and the conduit are configured to receive one or more surgical sutures therethrough. The graft is electrospun from one or more biocompatible polymers.

In some embodiments of the graft, the one or more biocompatible polymers comprise a first polymer co-electrospun with a second polymer.

In some embodiments of the graft, the one or more biocompatible polymers comprise polyglycolic acid (PGA) and poly(lactide co-caprolactone) (PLCL).

In some embodiments of the graft, a width of the conduit and a width of the aperture are each in a range from about 0.5 mm to about 3 mm.

In some embodiments of the graft, a length of the graft is from about 5 mm to about 100 mm and a width of the graft is from about 5 mm to about 100 mm.

In one embodiment, the present disclosure is directed to a method of fabricating a graft. The method includes receiving a sheet electrospun from one or more biocompatible polymers; forming an aperture in the electrospun sheet, where the aperture is configured to receive one or more surgical sutures therethrough; placing a rod against the electrospun sheet; folding a second portion of the electrospun sheet over the rod and a first portion of the electrospun sheet; forming a conduit around the rod from corresponding portions of the first portion and the second portion, where the conduit is configured to receive the one or more surgical sutures therethrough; and securing the first portion to the second portion.

In some embodiments of the method, the aperture is formed by one or more of cutting, ablating, or mechanically punching.

In some embodiments of the method, the conduit is formed by heat sealing the second portion to the first portion around the rod.

In some embodiments of the method, the first portion and the second portion are secured via at least one of heat sealing or a biocompatible adhesive.

In some embodiments of the method, the one or more biocompatible polymers comprise a first polymer co-electrospun with a second polymer.

In some embodiments of the method, the one or more biocompatible polymers comprise PGA and PLCL.

In some embodiments of the method, a width of the conduit and a width of the aperture are each in a range from about 0.5 mm to about 3 mm.

In some embodiments of the method, a length of the graft is from about 5 mm to about 100 mm and a width of the graft is from about 5 mm to about 100 mm.

In one embodiment, the present disclosure is directed to an electrospun structure comprising: one or more sheets electrospun from one or more biocompatible polymers, the one or more sheets heat sealed to form a heat sealed peripheral edge defining an interior compartment configured to hold at least one of a pharmaceutical, a biologic material, or a medical device.

In some embodiments of the electrospun structure, the one or more biocompatible polymers comprise a first polymer co-electrospun with a second polymer.

In some embodiments of the electrospun structure, the one or more biocompatible polymers comprise PGA and PLCL.

In some embodiments of the electrospun structure, the heat sealed peripheral edge forms an enclosed interior compartment.

In some embodiments of the electrospun structure, the heat sealed peripheral edge defines an opening to the interior compartment.

In some embodiments of the electrospun structure, the electrospun structure further comprises an antibiotic, an analgesic, demineralized bone matrix (DBM), thrombin, platelet rich plasma (PRP), adipose tissue, a growth factor, autologous tissue, allograft tissue, xenograft tissue, or any combination thereof disposed within the interior compartment.

### BRIEF DESCRIPTION OF THE DRAWINGS

FIG. 1 shows a perspective view of an electrospun onlay configured for suture management in accordance with an embodiment of the present disclosure.

FIG. 2 shows a diagram of a pair of electrospun onlays used in a rotator cuff repair in accordance with an embodiment of the present disclosure.

FIG. 3A shows an elevational view of an electrospun sheet for constructing an onlay in accordance with an embodiment of the present disclosure.

FIG. 3B shows an elevational view of the electrospun sheet folded to form the onlay in accordance with an embodiment of the present disclosure.

FIG. 3C shows a perspective view of the electrospun sheet of FIG. 3A in accordance with an embodiment of the present disclosure.

FIG. 4A shows an elevational view of an electrospun sheet for constructing an onlay in accordance with an embodiment of the present disclosure.

FIG. 4B shows an elevational view of the electrospun sheet folded to form the onlay in accordance with an embodiment of the present disclosure.

FIG. 4C shows a perspective view of an electrospun sheet for constructing an onlay in accordance with an embodiment of the present disclosure.

FIG. 4D shows a perspective view of the electrospun sheet folded to form the onlay in accordance with an embodiment of the present disclosure.

FIG. 5 shows a diagram of a heat-sealed electrospun pouch in accordance with an embodiment of the present disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the term “biocompatible” refers to materials exhibiting non-harmful compatibility with living tissue. Biocompatibility is a broad term that describes a number of materials, including bioinert materials, bioactive materials, bioabsorbable materials, biostable materials, biotolerant materials, or any combination thereof.

As used herein, the term “bioresorbable” refers to materials that are biodegradable or are naturally absorbed by a body over time.

As used herein, the singular forms “a,” “an,” and “the” include plural references, unless the context clearly dictates otherwise. Thus, for example, reference to a “protein” is a reference to one or more proteins and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “subject” includes, but is not limited to, humans and non-human vertebrates such as wild, domestic, and farm animals.

### Electrospinning

Electrospinning is a method which may be used to process a polymer solution into a structure, such as a fiber. Generally speaking, a conventional electrospinning setup includes applying a high voltage to a spinneret (e.g., a needle or a wire) through which a polymer solution or melt is driven (e.g., at a rate of 0.1-50 mL/hr). Various types of polymer solution configurations, which can include a polymer dissolved in a solvent, are described below. As the polymer solution flows through the spinneret, it is electrostatically driven to form a Taylor cone. A jet of the polymer solution ejects from the Taylor cone towards a receiving surface that is positioned a distance from the spinneret. This receiving surface may or may not be charged with a high voltage. As the jet travels through the air to the receiving surface, it destabilizes, which causes the polymer within the jet to elongate and thin while the solvent simultaneously evaporates. This leads to the formation of solid polymer nanofibers which deposit on the receiving surface. In embodiments where the diameter of the resulting fiber is on the nanometer scale, the fiber may be referred to as a nanofiber. Fibers may be formed into a variety of shapes by using a range of receiving surfaces, such as mandrels, molds, or collectors. The resulting electrospun structures may be used in many applications, including as emulsifying and stabilizing agents for food products.

Electrospinning methods may involve spinning a structure (e.g., a fiber) from a polymer solution by applying a high DC or AC voltage potential between a polymer injection system and a receiving surface. In some embodiments, a charge may be applied to one or more components of an electrospinning system. In some embodiments, a charge may be applied to the receiving surface, the polymer injection system, the polymer solution, or combinations or portions thereof. Without wishing to be bound by theory, as the polymer solution is ejected from the polymer injection system, it is thought to be destabilized due to its exposure to a charge. The destabilized solution may then be attracted to a charged receiving surface. As the destabilized solution moves from the polymer injection system to the receiving surface, its solvents may evaporate, and the polymer may stretch, leaving a long, thin fiber that is deposited onto the receiving surface. The polymer solution may form a Taylor cone as it is ejected from the polymer injection system and exposed to a charge. Further, polymers can be electrospun in a variety of different structures, including fibers, fibrous scaffolds, strips, patches, sheets, or shapes corresponding to anatomical structures. Still further, the structures can be electrospun using one or multiple polymers.

In some embodiments, multiple polymer types can be electrospun with each other to form structures in a process referred to as “co-electrospinning.” In co-electrospinning, two or more polymer solutions (containing the same or different polymer types) are ejected from different outlets and simultaneously electrospun with each other to form the resultant structure. Co-electrospinning creates different fibers formed from the different polymer solutions that are intertwined with each other. The co-electrospun polymers can be spun from the same or different polymer solutions. The co-electrospun polymer types can have the same or different degradation rates.

In some embodiments, multiple polymer types can be electrospun with each other to form structures in processes referred to as “coaxial electrospinning” or “multiaxial electrospinning.” In coaxial or multiaxial electrospinning, two or more polymer solutions (containing the same or different polymer types) are ejected from the same outlet and electrospun with each other to form the resultant structure. Coaxial or multiaxial electrospinning creates a single fiber composed of the different polymer types that has a core-shell structure. The coaxially or multi-axially electrospun polymer types can have the same or different degradation rates.

In some embodiments, the co-electrospinning and coaxial or multiaxial electrospinning techniques described above could also be used in combination with each other. For example, coaxial polymers or fibers could be co-electrospun with each other. Accordingly, the various techniques described above can be used to create electrospun structures having various degradation rates for the constituent polymers.

### Polymer Injection System

A polymer injection system may include any system configured to eject some amount of a polymer solution into an atmosphere to permit the flow of the polymer solution from the injection system to the receiving surface. In some embodiments, the polymer injection system may deliver a continuous or linear stream with a controlled volumetric flow rate of a polymer solution to be formed into a structure (e.g., a fiber). In some embodiments, the polymer injection system may deliver a variable stream of a polymer solution to be formed into a fiber. In some embodiments, the polymer injection system may be configured to deliver intermittent streams of a polymer solution to be formed into multiple fibers.

The polymer injection system could include a variety of different components and/or mechanisms for ejecting the polymer solution, including needle-based injection systems (e.g., a syringe) or needleless injection systems (e.g., a rotating surface, a wire-based injection system, or any type of free-surface with an applied electrical charge). In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with one or more syringes containing one or more different polymer solutions.

The needless injection systems could include a variety of different configurations, all of which are within the scope of the present disclosure. In some embodiments, the polymer injection system could include a rotating drum or any type of surface that dips into the polymer solution and ejects the solution as the drum or surface rotates. In some embodiments, the polymer injection system could include a wire-based electrospinning system (e.g., the NS 8S1600U electrospinning production line available from ELMARCO®).

In some embodiments, the polymer injection system could include a slit injector system. A slit injector essentially functions as a hybrid between needle-based and needless electrospinning designs. A slit injector includes an extended slit that is positioned opposite the receiving surface. In operation, the electrospun polymer solution is ejected longitudinally along the slit and received by the receiving surface. Slit injector systems could be used to form conventional electrospun fibers, as well as core-shell fibers and other fiber configurations.

In some embodiments, the polymer solution may be ejected from the polymer injection system at a flow rate per needle of less than or equal to about 5 mL/h. Some non-limiting examples of flow rates per needle may include about 0.1 mL/h, about 0.5 mL/h, about 1 mL/h, about 1.5 mL/h, about 2 mL/h, about 2.5 mL/h, about 3 mL/h, about 3.5 mL/h, about 4 mL/h, about 4.5 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any ranges between any two of these values, including endpoints.

As the polymer solution travels from the polymer injection system toward the receiving surface, the diameter of the resulting fibers may be in the range of about 0.1 μm to about 10 μm. Some non-limiting examples of electrospun fiber diameters may include about 0.1 μm, about 0.2 μm, about 0.5 μm, about 1 μm, about 2 μm, about 5 μm, about 10 μm, about 15 μm, about 20 μm, or ranges between any two of these values, including endpoints.

### Electrospun Polymer Solution

In some embodiments, the polymer injection system may be filled with a solution from which structures, such as fibers, can be electrospun. Various components of the solution are described below.

In some embodiments, the electrospun solution can comprise a protein. In some embodiments, the protein could include an animal-derived or a non-animal-derived protein. As used herein, a “non-animal-derived” protein refers to a protein derived from natural (i.e., non-synthetic) sources not including animals, such as plants, fungi, bacteria, or algae. In some embodiments, the protein could include a legume-derived, fruit-derived, grain-derived, fungi-derived, or animal-derived protein. The protein can include, for example, zein or another corn-derived protein, a pea-derived protein, a wheat-derived protein, an oat-derived protein, a whey-derived protein, a soy-derived protein, or combinations thereof. The proteins described herein can include isolates, concentrates, hydrolysates, micronized proteins, functionalized proteins, solubilized proteins, base-treated proteins, and/or enzyme-hydrolyzed proteins. For example, a “pea-based protein” or a “pea-derived protein” would include any protein that is isolated, concentrated, hydrolyzed, micronized, functionalized, solubilized, base-treated, enzyme-hydrolyzed, or otherwise developed from a pea plant.

In some embodiments, the electrospun solution can comprise a biological polymer, such as polysaccharides. The polysaccharides can include, for example, pullulan, dextran, dextrin, maltodextrin, pectin, agarose, chitin, cellulose, xylose, xanthan gum, welan gum, gellan gum, alginate, or combinations thereof. In some embodiments, the polysaccharide can include any polysaccharide that is configured to function as a reducing polysaccharide in a glycation reaction.

In a preferred embodiment, the electrospun solution can include at least one protein and at least one polysaccharide. In some embodiments, the electrospun solution can include combinations of multiple proteins and/or multiple biological polymers. Such formulations can be beneficial because incorporating multiple proteins and/or multiple biological polymers into the polymer solution can improve the production of the conjugated fibers, and also provide desirable chemical functionality characteristics. For example, one type of protein could be selected for its functionality or downstream performance (e.g., improved emulsification) and a second type of protein could be selected for its processing speed (but, in some cases, provide comparatively less functionality than the other protein type). Accordingly, a blend or combination of the multiple protein types could provide a beneficial mix of both of these properties. Likewise, for example, one type of biological polymer could be selected for its functionality or downstream performance (e.g., improved emulsification) and a second type of biological polymer could be selected for its processing speed (but, in some cases, provide comparatively less functionality than the other biological polymer type). Accordingly, a blend or combination of the multiple biological polymer types could provide a beneficial mix of both of these properties.

In some embodiments, the electrospun solution can include a processing aid or polymer in combination with the biological polymer. In some embodiments, the polymer may be a fluid formed into a polymer liquid by the application of heat. The polymer can include natural, synthetic, or semi-synthetic polymers such as, without limitation, alginate, fibronectin, hyaluronic acid, poly(ethylene oxide) (PEO), polyvinyl pyrrolidone, dextran, saccharide, cellulose, chitosan, gelatin, collagen, polyvinyl alcohol (PVA), Eudragit, polyester, polymethylmethacrylate, polyacrylonitrile, silicone, polyether ether ketone, polyether imide, polyamide, polyether sulfone, polysulfone, polyvinyl acetate (PVAc), polycaprolactone (PCL), polylactic acid (PLA), poly(lactide co-caprolactone) (PLCL), polylactide co-glycolide (PLGA), polyglycolic acid (PGA), polyglycerol sebacic, polydiol citrate, polyhydroxy butyrate, polyether amide, polydioxanone (PDO), polytrimethylenecarbonate (TMC), copolymers thereof, enantiomers, and combinations or derivatives thereof. In some embodiments, the polymer may include a water-soluble polymer. It may be understood that the polymer may also include a combination of synthetic polymers and naturally occurring polymers in any combination or compositional ratio. In general, the addition of one or more polymers to the electrospun solution can improve the ability to produce the protein-polysaccharide conjugates at large scale as compared to electrospinning a solution exclusively containing the aforementioned proteins and polysaccharides. In particular, natural materials, including proteins and polysaccharides, typically need to be electrospun with lower throughput and with more process restrictions than synthetic polymers because electrospinning natural materials at the same throughput as synthetic polymers can result in process errors. However, the addition of even a small proportion of a synthetic polymer as a processing aid can dramatically improve the process throughput and stability of the resulting electrospun construct. For example, a polymer can act as a surfactant (e.g. Triton or poloxamers), thereby helping to achieve a more desirable surface tension of the solution as it is electrospun. As another example, the polymer can act as a binding agent, thereby increasing molecular entanglement of the solution components and helping to achieve a desirable viscoelasticity of the solution. As another example, the polymer or molecule may affect the release rate of the ingredients in the fibers. For example, an excipient may be used to increase the release of an active pharmaceutical ingredient. Or conversely, a cyclodextrin may be used to entrap an ingredient and prevent it from leaching out.

In some embodiments, the biological polymer may be present in an amount of about 1 wt % to about 30 wt % based on the weight of the polymer solution. In some non-limiting examples, the polymer may be present in the amount of, for example, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, or ranges between any two of these values, including endpoints.

In some embodiments, the polymer solution may comprise a solvent. The solvents can include organic or inorganic solvents. In some embodiments, the solvent may comprise, for example, acetone, dimethylformamide, dimethylsulfoxide, N-methylpyrrolidone, N,N-dimethylformamide, acetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane (DCM), water, alcohols, ionic compounds, or combinations thereof. Non-limiting examples of alcohols include ethanol, isopropanol, butanol, and the like. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

### Applying Charges to Electrospinning Components

In an electrospinning system, a charge may be applied to one or more components, or portions of components, such as, for example, a receiving surface, a polymer injection system, a polymer solution, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to the polymer solution, or portions thereof. In some embodiments, a negative charge may be applied to the polymer solution, or portions thereof. In some embodiments, the polymer solution, or portions thereof, may be grounded. In some embodiments, a positive charge may be applied to the receiving surface, or portions thereof. In some embodiments, a negative charge may be applied to the receiving surface, or portions thereof. In some embodiments, the receiving surface, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −100 kV to about 100 kV, including endpoints. In some non-limiting examples, the charge applied to any component of the electrospinning system, or portions thereof, may be about −100 kV, about −75 kV, about −50 kV, about −30 kV, about −25 kV, about −15 kV, about −10 kV, about −5 kV, about −3 kV, about −1 kV, about −0.01 kV, about 0.01 kV, about 1 kV, about 5 kV, about 10 kV, about 12 kV, about 15 kV, about 20 kV, about 25 kV, about 30 kV, about 50 kV, about 60 kV, about 75 kV, about 100 kV, or any range between any two of these values, including endpoints. In some embodiments, any component of the electrospinning system, or portions thereof, may be grounded.

### Receiving Surface Movement During Electrospinning

During electrospinning, in some embodiments, the receiving surface may move with respect to the polymer injection system. In some embodiments, the polymer injection system may move with respect to the receiving surface. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the receiving surface may be in contact with or mounted upon a support structure that may be moved using one or more motors or motion control systems. The pattern of the electrospun structure deposited on the receiving surface may depend upon one or more motions of the receiving surface with respect to the polymer injection system. In some embodiments, the receiving surface may be configured to rotate about its long axis. In one non-limiting example, a receiving surface having a rotation rate about its long axis that is faster than a translation rate along a linear axis may result in a nearly helical deposition of an electrospun fiber, forming windings about the receiving surface. In another example, a receiving surface having a translation rate along a linear axis that is faster than a rotation rate about a rotational axis may result in a roughly linear deposition of an electrospun fiber along a linear extent of the receiving surface. In some embodiments, the electrospinning system could include a roller electrospinning system.

In some embodiments, the receiving surface of the electrospinning system can be moved (e.g., rotated) in a continuous or discontinuous manner. In one embodiment, the receiving surface can include a cylindrical mandrel that is rotated about a central axis. In some embodiments, the receiving surface can include metal fibers (e.g., steel wool or steel mesh), an organic woven fabric, an organic non-woven fabric, a synthetic woven fabric, a synthetic non-woven fabric, paper, a paper-like substrate, impregnated paper, polymer film, or combinations thereof. Receiving surfaces including such substrates can improve the ability to mass produce the conjugated fibers compared to conventional methods.

### Electrospun Onlay Scaffolds Having Improved Suture Management

Described herein are biocompatible electrospun scaffolds and methods of improving bone-soft tissue healing using the same. The biocompatible scaffolds described herein could include bioresorbable or biostable scaffolds, i.e., scaffolds that are constructed from bioresorbable or biostable materials. Biocompatible scaffolds are often used in various surgical procedures, including rotator cuff repairs. In such procedures, the scaffolds are placed between the bone and the tendon in order to provide a microporous, microfiber scaffold matrix to support tissue growth in order to facilitate tendon-bone attachment. Various examples of electrospun biocompatible scaffolds and techniques for using such scaffolds for improving bone-soft tissue healing are described in U.S. patent application Ser. No. 15/887,301, now U.S. Pat. No. 10,898,608, titled METHODS OF IMPROVING BONE-SOFT TISSUE HEALING USING ELECTROSPUN FIBERS, filed Feb. 2, 2018, which is hereby incorporated by reference herein in its entirety.

Biocompatible scaffolds are generally surgically secured in place between the bone and tendon using suture anchors. However, suture anchors can be cumbersome to manage intraoperatively by the surgeon, especially during arthroscopic procedures. For example, an arthroscopic rotator cuff repair procedure generally uses two accesses (one port for a camera and one port for a surgical tool) and typically requires four sutures to be applied per suture anchor (i.e., up to eight sutures total for a rotator cuff repair). Further, the joint is continually perfused with water during the arthroscopic procedure. Therefore, surgeons are forced to manage up to eight total sutures, through a single access port, that are being buffeted by the perfusion water as the surgeon is attempting to secure the sutures in place. Managing these sutures can be highly challenging for even a skilled surgeon. To solve these and other problems, described herein are biocompatible electrospun scaffolds that are designed to aid surgeons in managing the placement of sutures during arthroscopic procedures. In some embodiments, a scaffold used for augmenting rotator cuff repairs and other such procedures can be referred to as a “graft,” an “onlay patch,” or an “onlay.” The various terms can be used interchangeably in this particular context. Further, the specific benefits of electrospun grafts, onlays, and/or scaffolds are described in more detail in U.S. Pat. No. 10,898,608.

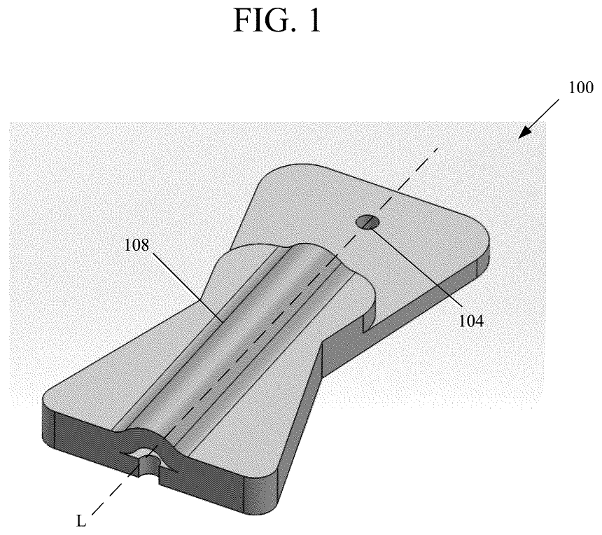

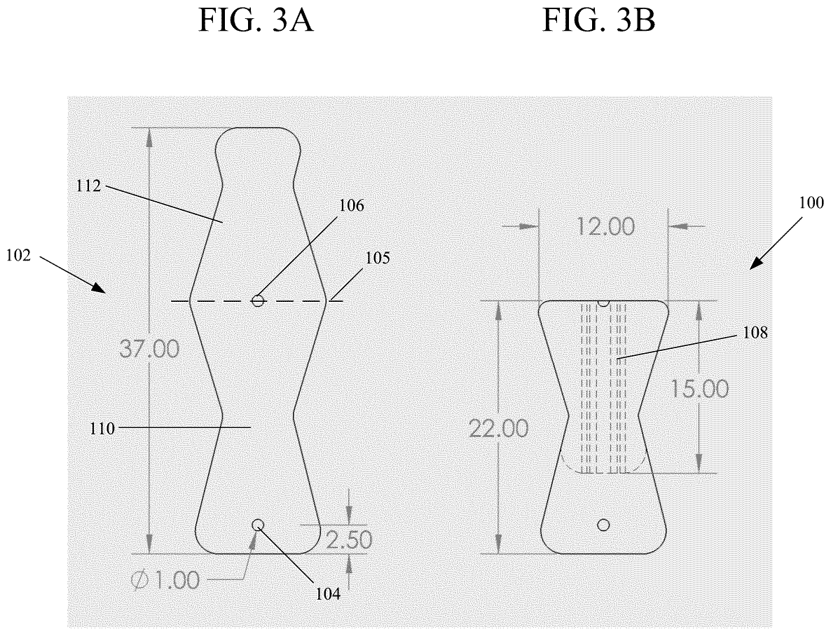

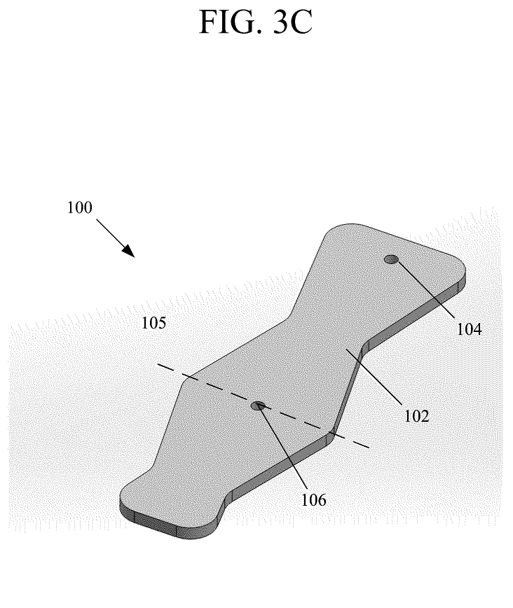

One embodiment of an electrospun onlay 100 is shown in FIG. 1A. The onlay 100 can include a conduit 108 and an aperture 104 aligned with the longitudinal axis L of the conduit 108. The conduit 108 and the aperture 104 can be sized or otherwise configured to receive a suture therethrough. In one embodiment, the diameter or width of the conduit 108 and/or the aperture 104 could be from about 0.5 mm to about 3 mm. In one illustrative embodiment, the diameter of the conduit 108 and/or aperture 104 could be about 1 mm. The longitudinal axis L of the conduit 108 is generally aligned with the aperture 104 so that one or more sutures can be threaded through the conduit 108 into the aperture 104 (and then secured to bone or tissue), as shown in FIG. 3, for example. This positioning of the aperture 104 and the onlay 108 can be particularly beneficial because it could prevent medial tears of the rotator cuff caused by pull-through by the sutures. Accordingly, the conduit 108 covers and maintains the sutures tightly against the onlay 100 (i.e., preventing the sutures from moving or becoming entangled during the procedure), thereby keeping the sutures organized and allowing the sutures to be more easily managed by the surgeon during a procedure. Further, including the conduit 108 for the onlay 100 improves the case which the onlay 100 can be intraoperatively secured in place, eliminates the need for secondary fixation methods and/or implants (which lowers risk factors associated with the surgical procedure because it limits the amount of surgical intervention that the surgeon must perform), helps distribute the load placed on the tendon (which minimize the amount of damage or trauma from the sutures), and facilitates optimal compression of the tendon. The onlay 100 can be electrospun from one or more polymers using any of the techniques described above. In one embodiment, the conduit 108 could include a generally tubular structure having open ends that is fully sealed along the edges of its sidewalls against the body of the onlay 100. In another embodiment, the conduit 108 be only partially sealed along the edges of its sidewalls against the body of the onlay 100, i.e., there could be gaps along the lateral edges of the sidewalls of the conduit 108.

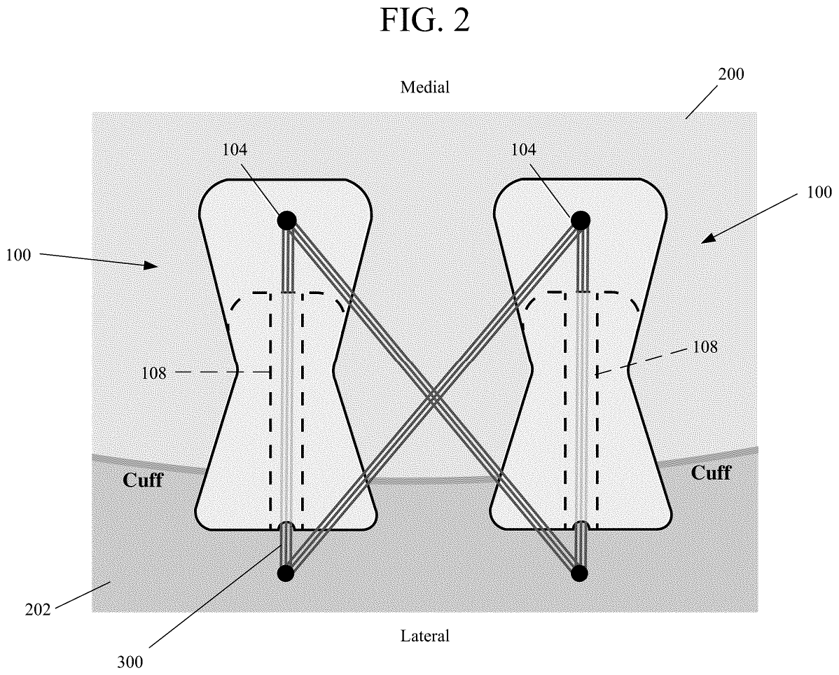

FIG. 2 illustrates a diagram demonstrating how onlays 100 can be used in a rotator cuff repair procedure. In a rotator cuff repair, onlays 100 are secured to the patient's tendon 200 and rotator cuff 202 via surgical sutures 300. In particular, one or more surgical sutures 300 are threaded through the patient's rotator cuff 202, through the conduit 108 of an onlay 100 to the suture aperture 104, and then through the tendon 200. Therefore, the onlay 100 is secured to both the tendon 200 and the rotator cuff 202. As shown in FIG. 2, the sutures 300 can be threaded from the tendon 200 back through the rotator cuff 202 and then back through a second onlay 100, as described above. Once secured in place, the electrospun onlays 100 are able to facilitate tissue growth for tendon-bone reattachment, as described above. The use of multiple onlays 100 in a rotator cuff repair procedure can be beneficial because they can provide additional reinforcement and allow for the sutures 300 to be secured in a more stabilized manner. Conventional onlays for rotator cuff repair procedures lack the conduits 108 or any other mechanism for keeping the sutures 300 organized during the procedure; therefore, the sutures 300 are fully exposed to the perfusion liquid, causing the sutures 300 to move constantly during the procedure until they are fully and tightly secured to the patient's anatomy. Intraoperatively managing multiple moving sutures 300 during a procedure can be highly challenging for a surgeon. Therefore, the onlays 100 described herein that include the conduits 108 for covering the sutures 300 and keeping the sutures 300 organized intraoperatively are highly beneficial because they increase the case with which the surgeon can perform the procedure. This can in turn facilitate improved patient outcomes, particularly improved healing and bone-tendon reattachment, due to the surgeon being able to better secure the sutures 300 to the patient's anatomy.

In various embodiments, the onlay 100 can be electrospun from any combination of the polymers described above. In embodiments where the onlay 100 is constructed from one or more biocompatible polymers, the onlay 100 will gradually degrade in vivo. In one embodiment, the onlay 100 can be constructed from two or more electrospun polymers. In one illustrative embodiment, the onlay 100 can be constructed from co-electrospun PGA and PLCL.

The electrospun onlay 100 can be fabricated in a number of different manners. In one embodiment, the onlay 100 can be formed from a single electrospun polymer sheet. FIGS. 3A and 3B illustrate a technique for forming the onlay 100 from a single electrospun polymer sheet. In one embodiment, the sheet 102 can be electrospun into the desired shape. In another embodiment, the sheet 102 can be cut from a large electrospun sheet or otherwise formed into the desired shape. As shown in FIG. 3A, an electrospun polymer sheet 102 can include a first portion 110 including the suture aperture 104 and a second portion 112. In the depicted embodiment, the first portion 110 has a dumbbell-like shape and the second portion 112 has a shape that generally corresponds to the first portion 110 when folded over the first portion 110 at the medial line 105, as shown in FIG. 3B. In this embodiment, the onlay 100 further includes a second aperture 106 that defines one of the open ends of the conduit 108. As described above, the sheet 102 can be electrospun from one or more polymers, including any of the polymers described above.

In operation, the second portion 112 of the electrospun polymer sheet 102 is folded over onto the first portion 110 at the medial line 105 that bisects the second aperture 106, as shown in FIG. 3B. The second portion 112 can be secured to the first portion 110 using a variety of different techniques, including heat sealing or biocompatible adhesives. In particular, the edges of the folded-over second portion 112 can be heat sealed to the corresponding underlying portions of the first portion 110 in order to secure the portions 110,112 together and form a unitary structure. This embodiment can be beneficial because the heat-sealed edges can provide stiffness to the onlay 100 that assists the onlay 100 in laying flat. It can be beneficial for the onlay 100 to lay flat in operation because the anatomy of the rotator cuff or shoulder is generally semi-spherical; accordingly, the ability for the onlay 100 to lay flat allows it to form to the curvature of the patient's anatomy. By precisely conforming to the patient's anatomy, the onlay 100 is prevented from snagging on other portions of the patient's anatomy and otherwise is placed in better communication with the patient's tissue to better facilitate healing.

The suture aperture 104 and the second aperture 106 can be formed in a variety of different manners, including cutting, ablating, or mechanically punching the electrospun onlay 100. The conduit 108 can be formed in a variety of different manners. In one embodiment, a rod can be placed through the second aperture 106 and laid across the first portion 110. As the second portion 112 is folded over the second portion 110 as described above, the second portion 112 can be pressed around the rod. Further, in one embodiment, heat sealing elements can be pressed around the rod, adhering the corresponding portions of the first and second portions 110, 112 to each other around the rod and thereby forming the conduit 108.

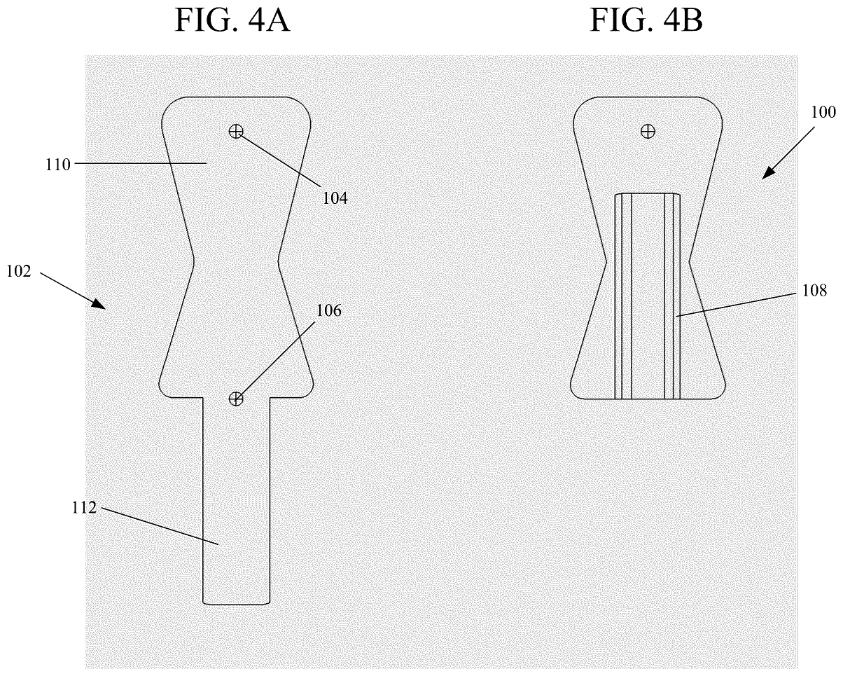

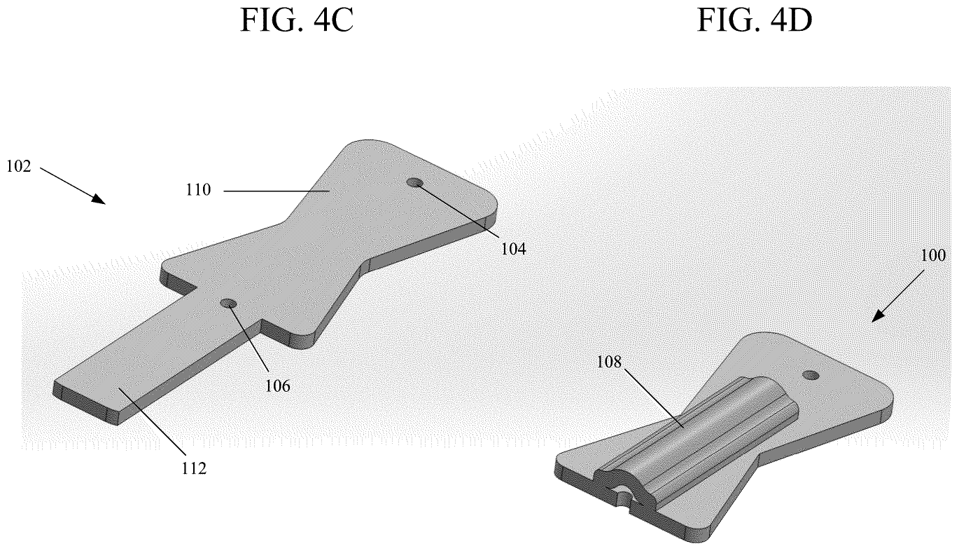

Although the second portion 112 of the sheet 102 is depicted in FIGS. 3A-3C as generally matching with the shape of the corresponding portions of the first portion 110 of the sheet, the second portion 112 can have a variety of different shapes. For example, in an alternative embodiment shown in FIGS. 4A-4D, the second portion 112 has a generally rectangular shape. In this embodiment, the onlay 100 can be constructed using the same techniques as described above for the embodiment shown in FIGS. 3A-3C.

In various embodiments, the length of the onlay 100 could be from about 5 mm to about 100 mm. In various embodiments, the width of the onlay 100 could be from about 5 mm to about 100 mm.

### Heat Sealed Electrospun Structures

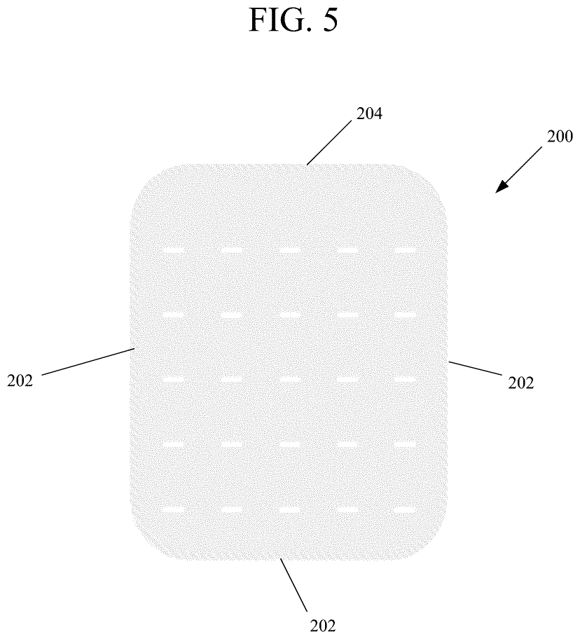

Biocompatible electrospun scaffolds can further be formed into a variety of different structures using the heat-sealing techniques described above. In some embodiments, electrospun structures can be formed by heat sealing one portion of an electrospun sheet to another portion of the electrospun sheet, such as is described above in the embodiments of the onlay 100. In some embodiments, electrospun structures can be formed by heat sealing corresponding portions of two or more electrospun sheets to each other. In one illustrative embodiment shown in FIG. 5, a heat-sealed electrospun pouch 200 can be formed by heat sealing portions of a first electrospun sheet to portions of a second electrospun sheet to form heat sealed edges 202 extending about a periphery of the pouch 200. In some embodiments, the pouch 200 can be sealed around its entire peripheral edge to form an enclosed structure. In some embodiments, such as is shown in FIG. 5, the pouch 200 can be heat sealed to leave one or more openings 204 through which material and/or objects can be inserted into the pouch 200 for storage therein. In any of these embodiments, the heat sealed edges 202 can define an interior compartment for holding various materials or objects within the pouch 200. The pouch 200 can be sized, shaped, or otherwise configured to hold a pharmaceutical, a biologic material, a medical device, or any combination thereof. In some embodiments, the material held within the pouch 200 could include antibiotics, analgesics, demineralized bone matrix (DBM), thrombin, platelet rich plasma (PRP), adipose tissue, growth factors, autologous tissue, allograft tissue, xenograft tissue, or any combination thereof. In various embodiments, the pharmaceutical, biologic material, and/or medical device could be completely enclosed within the pouch by heat sealing the edges 202 therearound or could be inserted within the pouch 200 through an opening 204.

The heat sealed electrospun structures described herein can be formed from one or more sheets electrospun from one or more biocompatible polymers, including any of the polymers described above. The biocompatible polymers can be electrospun into sheets and/or structures using any of the techniques described above, including co-electrospinning. In some embodiments, the electrospun sheets can include one layer of electrospun polymers. In other embodiments, the electrospun sheets can include two or more layers of electrospun polymers. In one illustrative embodiment, the pouch 200 illustrated in FIG. 5 could be constructed from two sheets that each include three layers of electrospun material.

As generally described above, the benefits of heat sealing the electrospun structure can include the additional stiffness provided by the heat sealed edges 202 to the overall structure, while also allowing the electrospun structure to lay flat. The heat sealed edges may form a pouch that contains a pharmaceutical compound, biological compound, and/or medical device and prevent it from moving or migrating from the implantation site. Additionally, the electrospun structure may provide a barrier layer to prevent host cells from infiltrating into the pouch to help protect the pouch contents.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1. A graft comprising:
an aperture; and
a conduit extending towards the aperture, wherein a longitudinal axis of the conduit is aligned with the aperture;
wherein the aperture and the conduit are configured to receive one or more surgical sutures therethrough;
wherein the graft is electrospun from one or more biocompatible polymers.

2. The graft of claim 1, wherein the one or more biocompatible polymers comprise a first polymer co-electrospun with a second polymer.

3. The graft of claim 1, wherein the one or more biocompatible polymers comprise PGA and PLCL.

4. The graft of claim 1, wherein a width of the conduit and a width of the aperture are each from about 0.5 mm to about 3 mm.

5. The graft of claim 1, wherein a length of the graft is from about 5 mm to about 100 mm and a width of the graft is from about 5 mm to about 100 mm.

6. The graft of claim 1, wherein:
the graft is formed from a sheet comprising the one or more electrospun biocompatible polymers; and
the conduit is formed via a first portion of the sheet folded over and secured to a second portion of the sheet.

7. The graft of claim 6, wherein the first portion of the sheet is secured to the second portion via heat sealing.

8. A method of fabricating a graft, comprising:
receiving a sheet electrospun from one or more biocompatible polymers;
forming an aperture in the electrospun sheet, wherein the aperture is configured to receive one or more surgical sutures therethrough;
placing a rod against the electrospun sheet;
folding a second portion of the electrospun sheet over the rod and a first portion of the electrospun sheet;
forming a conduit around the rod from corresponding portions of the first portion and the second portion, wherein the conduit is configured to receive the one or more surgical sutures therethrough;
securing the first portion to the second portion.

9. The method of claim 8, wherein the aperture is formed by one or more of cutting, ablating, or mechanically punching.

10. The method of claim 8, wherein the conduit is formed by heat sealing the second portion to the first portion around the rod.

11. The method of claim 8, wherein the first portion and the second portion are secured via at least one of heat sealing or a biocompatible adhesive.

12. The method of claim 11, wherein the heating sealing increases a stiffness of the electrospun sheet at locations where the first portion is heat sealed to the second portion.

13. The method of claim 8, wherein the one or more biocompatible polymers comprise a first polymer co-electrospun with a second polymer.

14. The method of claim 8, wherein the one or more biocompatible polymers comprise PGA and PLCL.

15. The method of claim 8, wherein a width of the conduit and a width of the aperture are each from about 0.5 mm to about 3 mm.

16. The method of claim 8, wherein a length of the graft is from about 5 mm to about 100 mm and a width of the graft is from about 5 mm to about 100 mm.

17. An electrospun structure comprising:
one or more sheets electrospun from one or more biocompatible polymers, the one or more sheets heat sealed to form a heat sealed peripheral edge defining an interior compartment configured to hold at least one of a pharmaceutical, a biologic material, or a medical device.

18. The electrospun structure of claim 17, wherein the one or more biocompatible polymers comprise a first polymer co-electrospun with a second polymer.

19. The electrospun structure of claim 17, wherein the one or more biocompatible polymers comprise PGA and PLCL.

20. The electrospun structure of claim 17, wherein the heat sealed peripheral edge forms an enclosed interior compartment.

21. The electrospun structure of claim 17, wherein the heat sealed peripheral edge defines an opening to the interior compartment.

22. The electrospun structure of claim 17, wherein the electrospun structure further comprises an antibiotic, an analgesic, demineralized bone matrix (DBM), thrombin, platelet rich plasma (PRP), adipose tissue, a growth factor, autologous tissue, allograft tissue, xenograft tissue, or any combination thereof disposed within the interior compartment.
